(19) United States
(12) Patent Application Publication
(10) Pub. No.: US 2014/0308293 A1
(43) Pub. Date: Oct. 16, 2014

(54) RECONSTITUTION METHOD FOR HIGH CONCENTRATION DRY PROTEIN FORMULATIONS

(71) Applicant: XSTALBIO LIMITED, Glasgow (GB)

(72) Inventors: Jan Vos, Glasgow (GB); Andrew John MacLeod, Glasgow (GB)

(73) Assignee: XSTABIO LIMITED, Glasgow (UK)

(21) Appl. No.: 14/367,715

(22) PCT Filed: Dec. 24, 2012
(86) PCT No.: PCT/GB2012/053266

§ 371 Date: Jun. 20, 2014

(30) Foreign Application Priority Data
Dec. 23, 2011 (GB) 1122430.0

(51) Int. Cl. A61K 9/00; A61K 38/38; A61K 31/716; C07K 16/00

(52) U.S. Cl. CPC A61K 9/0095; C07K 16/00; A61K 38/385; A61K 31/716
USPC 424/142.1; 514/15.2; 514/58

(57) ABSTRACT

The present invention relates to the provision of a novel method for the reconstitution of dry formulations comprising biomolecules, and in particular, to dry protein formulations, and to pharmaceutical or veterinary products suitable for parenteral administration containing reconstituted formulations prepared according to the novel method of the invention.

### FIELD OF THE INVENTION

The present invention relates to the provision of a novel method for the reconstitution of dry formulations comprising biomolecules and in particular to dry protein formulations, and to pharmaceutical or veterinary products suitable for parenteral administration containing reconstituted formulations prepared according to the novel method of the invention.

### BACKGROUND OF THE INVENTION

As biomolecules are increasingly targeted as potential active pharmaceutical ingredients (APIs) within the bio-pharmaceutical industry, suitable formulations to enable the effective delivery of such materials are also required. Many biomolecules are initially formulated in the dry state for reconstitution prior to parental administration as solutions. However for many biomolecule-containing formulations, reconstitution is problematical, either due to the nature of the biomolecule itself, for example voluminous dry powders of biomolecules, or from aspects of the formulation, for example the desired concentration level, levels of foam produced or from inconsistencies between reconstituted formulations.

Therapeutic proteins such as monoclonal antibodies are important biomolecular drugs for the bio-pharmaceutical industry and there are many therapeutic proteins in development, targeted at a wide range of indications. Typically marketed therapeutic proteins are administered parentally as solutions and treatment may be administered to a subject in hospital via infusion or via injection from a healthcare professional or else be self-administered.

Therapeutic proteins that show poor stability in solution are often stabilized in the dry state. The stabilizing effects may vary from protein to protein but can include reducing mobility, increasing conformational stability and preventing or reducing water catalyzed degradation pathways.

When proteins are stored in the dry state as a dry protein formulation, such as a lyophilized powder or cake they most commonly need to be redissolved back into an aqueous diluent before they can be administered to the patient as a solution. The formation of a protein solution on solubilizing a dry protein formulation by addition of a suitable quantity of a diluent, such as water for injection, is generally termed reconstitution. The reconstitution process, beginning with addition of diluent, typically transforms a dry protein formulation from a powder or cake into a solution of the protein. Preferably on completion of the reconstitution process the formed protein solution will be optically clear or else opalescent, but it should not contain any visible particles.

The presence of visible or sub-visible particles may be indicative that degradation processes leading to the formation of protein aggregates have occurred during one or more of the product manufacturing steps, such as filling, freezing, or drying, or during post-manufacture shipping and storage, or else during the reconstitution process itself. It should be noted that great care is taken during the development of marketed therapeutic proteins products to ensure that the risk of producing particles during the manufacture, shipping and storage is minimized. This is because particles present in an administered solution of a therapeutic protein, particularly those that contain denatured protein, are likely to significantly increase the risk of a patient developing an undesirable immune response towards the protein drug. Problems with immunogenicity may include generation of anti-drug antibodies that neutralize or enhance the clearance of the therapeutic protein or else lead to accumulation of the drug. Thus there is a need for a process for the reconstitution of dry biomolecular formulations, and in particular dry protein formulations, which provides optically clear solutions, without visible particles, and preferably with minimal formation of foam.

The specific process of reconstituting a dry protein formulation is known to carry risks of causing aggregation of the protein which may result in formation of visible or sub-visible particles. Thus, it is well known in the art that shaking or vigorously agitating a protein/diluent mixture can result in a poorly reconstituted product possibly due to shear stresses and/or the production of bubbles which can both cause protein denaturation or unfolding. Thus, commonly the reconstitution instructions provided in the drug product insert of a therapeutic protein, specifically state “do not shake”. Protein solutions that have been shaken or agitated too vigorously typically contain a layer of persistent foam in which air bubbles, released during reconstitution of the dry protein formulation powder or else formed directly by shaking or agitation. It is thought that these bubbles may be stabilized by the presence of denatured protein. Thus there is a need for a process for an improved method for the reconstitution of dry biomolecular formulations, and in particular dry protein formulations, which mitigates the risk of producing visible and sub-visible particle formation.

Whilst the reconstitution instructions provided for dry protein formulations vary in detail from protein to protein the following examples of reconstitution steps-in-common are to be found in the product inserts of the following drugs Synagis® (palivizumab), Herceptin® (trastuzmab), Fuzeon® (enfuvirtide), and Xolair® (omalizumab) which are supplied as dry products in vials: manual reconstitution; swirling (gentle) or rolling (gentle); no shaking; avoidance of foam; clear or opalescent solutions; no particulates.

Thus, the reconstitution of dry powder formulations of biomolecules and therapeutic protein is typically carried out by hand and relies on the skill and experience of the responsible person to ensure the process is reproducibly carried out without degradation of the protein. In order to achieve full reconstitution in a reasonable time, such as less than 30 minutes, it is often stated that it is necessary to “gently swirl the vial”. This is both time-consuming and subjective, and requires the administering person such as the physician to spend time in advance of treating the patient preparing the medicament. Further, if this is done incorrectly it can lead to the formation of “excessive foaming” with the possibility that some small fraction of the protein has been degraded. The person responsible for reconstituting a dry protein formulation has therefore to apply the appropriate technique to achieve gentle swirling while also judging whether or not the level of foam produced is excessive or not. Furthermore, if on administration a particular therapeutic protein solution does generate an immune response there is a risk that the patient will henceforth no longer obtain any therapeutic benefit from that drug. In addition, as reconstituted solutions are not typically stored, if treatment is delayed the drug may not be suitable for later-use and as such has been wasted. Thus there is a need for a process for an improved method for the reconstitution of dry biomolecular formulations, and in particular dry protein formulations, which provides reconstituted formulations suitable for administration to a patient, which can be reconstituted more quickly to reduce wastage, can be reconstituted via a more regulated method to minimize person-to-person differences, and for proteins in particular can be reconstituted with reduced risk of foaming.

The production of foam on reconstitution of dry therapeutic protein formulations or other therapeutic biomolecule formulations may also cause difficulties with the double blinding process typically used in clinical trials for new drugs. It is difficult to reproduce the same level and/or consistency of foam in reconstituted placebo formulations as in the reconstituted active drug formulations and so a skilled person administering the drug may be able to recognize which patients are receiving placebo and which are receiving drug. A reconstitution method that results in minimal or no foaming on reconstitution of dry biomolecule formulations could therefore provide clear benefits by facilitating more effective blinding of clinical trials.

There is therefore a clear need to develop improved reconstitution methods for dry biomolecule containing formulations, and for dry protein formulations in particular that reduce or obviate the risk of inadvertently administering a poorly reconstituted therapeutic biomolecule, particularly a protein.

Voluminous dry formulations comprising biomolecules present particular challenges for reconstitution when high concentrations of reconstituted biomolecule containing solution are required. The need for new methods for reconstituting dry protein formulations in particular becomes significantly greater when considering high protein concentration solutions such as those required for delivery of doses of greater than 100 mg of a protein drug in a single 1 ml injection. High concentration protein solutions are inherently more prone to problems arising from reversible and irreversible association processes and these may provide pathways to the formation of soluble and insoluble aggregates particularly after storage for long time periods, or following freeze-thaw cycles. By storing the protein in the dry state and reconstituting it to form a high concentration solution, only at the time of use, problems caused by slow aggregate formation may be prevented or alleviated. However, reconstitution of dry protein formulations to high concentration is not straightforward. This is because reconstitution times are generally found to increase non-linearly with protein concentration. As illustrated by FIG. 3A of application WO2011/017070 published on 10 Feb. 2011, relating to the reconstitution times for a variety of different dry protein formulations, the reconstitution times for a 50 mg/ml protein solution lie in the range of from 25 seconds to 11 minutes 30 seconds, for a 70 mg/ml protein solution reconstitution times lie in the range of from 11 seconds to 22 minutes and to prepare a 100 mg/ml protein solution reconstitution times lie in the range 5 minutes 40 seconds to 90 minutes. The reconstitution time to prepare a 100 mg/ml protein solution is about 5 to 30 times greater than for a 50 mg/ml protein solution.

This phenomenon of much greater reconstitution times being required for high concentration of biomolecule and proteins in particular may present particular difficulties when preparing biomolecule/protein solutions for administration to a patient. Vials that need to be swirled by hand, either continuously or intermittently, may take up unacceptable amounts of a medical practitioner's time. Further, the greater the ratio of powder to diluent the greater the skill level required to wet the powder without it sticking to the side of the vial. As the concentration of dissolved powder i.e. protein increases, the solution will also become more viscous and hence more energy needs to be expended to mix the solution while at the time great care needs to be taken not to over agitate the solution cause foaming. Hence, using current reconstitution methods the potential for foaming and the resultant risks of protein degradation become even greater at higher protein concentration.

There is therefore a need for a method or methods that can achieve reconstitution of a dry biomolecule formulation, to high biomolecule concentration, and particularly dry protein formulation to high protein concentration in a reduced/reasonable time, without formation of foam and with minimal manual intervention.

To be suitable for parenteral administration high biomolecule, or protein concentration solutions also need to comprise only pharmaceutically acceptable excipients and to fulfill other criteria such as retention of protein integrity, syringability and acceptable osmolality for injection. There is therefore also a need for methods that can produce stable dry powder formulations that can be reconstituted in a reasonable time, without formation of foam and with minimal manual intervention and which produce solutions containing protein of high integrity, with good syringability and acceptable osmolality. It is an object of the present invention to provide methods for the reconstitution of dry biomolecule formulations that reduce or obviate the risk of inadvertently administering a poorly reconstituted therapeutic biomolecule; produce stable dry powder formulations; that can be reconstituted in a reasonable time; that can be reconstituted without formation of foam; that can be reconstituted with minimal manual intervention; produce protein solutions containing protein of high integrity; that produce solutions having good syringability; that produce solutions having acceptable osmolality. It is also an object of the present invention to provide methods that can achieve reconstitution of a dry biomolecule formulation to a highly concentrated biomolecule solution in a reduced/reasonable time, without formation of foam and with minimal manual intervention.

### SUMMARY OF THE INVENTION

The present invention provides a method for reconstitution of dry formulations comprising biomolecules comprising:
i) transfer of a dry formulation comprising biomolecule(s) into a suitable reconstitution vessel, or preparation of such a dry formulation within a suitable reconstitution vessel;
ii) addition of a suitable quantity of an aqueous diluent to the reconstitution vessel; and
iii) centrifugation of the reconstitution vessel at a suitable relative centrifugal force for sufficient time to obtain complete or near reconstitution of said dry formulation into the aqueous diluent and to produce a solution that exhibits minimal or no foaming;
   wherein the order of steps (i) and (ii) may be reversed or combined providing for the transfer of diluent to the vessel followed by addition of the dry formulation, and providing for the transfer of a preformed mixture of a dry formulation compromising biomolecules and diluent to the vessel.

Where a dry formulation comprising biomolecules is added to the diluent in the reconstitution vessel, the present invention provides a method for the reconstitution of dry formulations comprising biomolecules comprising:
i) addition of a suitable quantity of an aqueous diluent to a suitable reconstitution vessel;
ii) transfer of a dry formulation comprising biomolecules into the reconstitution vessel and
iii) centrifugation of the reconstitution vessel at a suitable relative centrifugal force for sufficient time to obtain complete or near complete reconstitution of said formulation into the aqueous diluent and to produce a solution that exhibits minimal or no foaming.

The applicant has found that the reconstitution method of the present invention provides reconstituted formulations having desirable optical properties, provides reconstituted formulations in less time than previously achievable, and provides reconstituted formulations with reduced variability than previously achievable.

The reconstituted solutions provided by the method of the present invention are preferably homogeneous, optically clear and free from visible particles.

Where the dry formulation comprising biomolecules is a protein formulation, the applicant has found that reconstituted formulations having desirable protein integrity, desirable optical properties, and desirable protein solution concentration levels can be prepared via the method of the present invention. Thus the present invention provides a method for reconstitution of dry formulations comprising proteins comprising:
(i) transfer of a dry formulation comprising proteins into a suitable reconstitution vessel, or preparation of said dry formulation within a suitable reconstitution vessel;
ii) addition of a suitable quantity of an aqueous diluent to the reconstitution vessel; and
iii) centrifugation of the reconstitution vessel at a suitable relative centrifugal force for sufficient time to obtain complete or near reconstitution of said dry formulation into the aqueous diluent and to produce a protein solution that exhibits minimal or no foaming;
   wherein the order of steps (i) and (ii) may be reversed or combined providing for the transfer of diluent to the vessel followed by addition of dry protein, and providing for the transfer of a preformed mixture of dry protein and diluent to the vessel.

Where the dry protein is added to the diluent in the reconstitution vessel the present invention provides a method for the reconstitution of dry formulations comprising protein comprising:
(i) addition of a suitable quantity of an aqueous diluent to a suitable reconstitution vessel;
(ii) transfer of dry formulation comprising protein into the reconstitution vessel; or preparation of said dry formulation within the reconstitution vessel; and
iii) centrifugation of the reconstitution vessel at a suitable relative centrifugal force for sufficient time to obtain complete or near complete reconstitution of the dry formulation into the aqueous diluent and to produce a protein solution that exhibits minimal or no foaming.

The reconstituted solutions provided by the methods of the present invention are preferably homogeneous, optically clear and free from visible particles.

### DESCRIPTION

The method of the present invention provides reconstitution of biomolecule solutions from dry formulations comprising biomolecules, and in particular reconstituted protein solutions from dry formulations comprising proteins, within highly desirable time-frames. According to the method of the present invention reconstituted biomolecule solutions including protein solutions can be typically be prepared in less than 60 minutes or 30 minutes, preferably between 10 and 15 minutes, more preferably between 5 and 10 minutes.

Dry formulations comprising biomolecules as defined herein include both dried formulations of pure biomolecules and dried formulations of mixtures of biomolecules. Formulations of mixtures of biomolecules may include complex mixtures that have been derived from cellular sources such as bacterial lysates that have been made into a dry formulation.

The applicants have found that the centrifugation method of the present invention may advantageously be applied to the reconstitution of many dry formulations comprising biomolecules to provide reduction of the time to achieve full reconstitution, the reduction or elimination of foaming, and/or the minimisation of manual input. For the avoidance of doubt, where the term biomolecule is used in a general sense herein said term specifically includes proteins in particular.

Example 1 hereinafter illustrates the rapid reconstitution time of the present method versus that achievable using conventional protocols.

Suitable dry formulations comprising biomolecules which may be reconstituted according to the method of the present invention include formulations of: proteins, polysaccharides, nucleic acids, lipids and peptides, natural biopolymers or synthetic polymers, and mixtures and combinations thereof. Suitable dry biomolecule formulations may also include acellular formulations, formulations containing live cells or killed cells, attenuated cells or lysed cells or else live or killed viruses. For the avoidance of doubt, where the term biomolecule is used in a general sense herein said term specifically includes proteins in particular.

Determination of whether any particular dry formulation comprising biomolecules may be reconstituted according to the method of the present invention may be achieved via simple experiment as detailed in Example 2. Example 2 additionally provides an illustration of how the potential advantages of any particular reconstituted solution may be gauged.

Optionally on removal of the reconstitution vessel from the centrifuge a gentle mixing process can be applied to the solution to ensure homogeneity. Such, gentle mixing will remove any concentration gradients that may exist following centrifugation and can be carried out with for example a vial or syringe, via gentle swirling or rolling between the hands at a slight angle for a short time, such as from 5 to 30 seconds. Thus, the applicant has additionally found that following preparation of the protein or other biomolecule solution via the present methods, and prior to either transfer of the solution from a vial to a syringe for delivery to a subject, or delivery to a subject via syringe, or transfer to a suitable container for storage, subjecting the reconstitution vessel to gentle rotation at a slight angle can prove advantageous for the provision of a solution having a consistent concentration throughout. An additional benefit of this post centrifugation step may be inclusion of any minimal amounts of dry protein or other biomolecule formulation which were retained on the sides of the vessel during transfer i.e. residual powder flecks.

Thus the present invention additionally provides a reconstitution method as defined hereinbefore which optionally includes the step wherein the resultant solution is subject to gentle mixing to remove any residual concentration differences.

As will be appreciated complete reconstitution, has been achieved when one skilled in the art judges that the dry formulation comprising biomolecules is has been fully reconstituted and no further action is required, in other words it is considered to be optically clear. A fully reconstituted sample will typically be a homogeneous solution or homogeneous dispersion. Near complete reconstitution will arise if one skilled in the art judges that the dry formulation comprising biomolecules is very close to complete reconstitution. For example, near complete reconstitution may occur if there remains a small amount of material which has not been fully reconstituted, or if the solution is not homogeneous. A small amount of material remaining, typically less than 5% or 1% of the total amount of the dry formulation originally present, may remain attached to the walls of the reconstitution vessel and may be above the level of the solution. Typically it is possible to achieve complete reconstitution of a sample that has reached near complete reconstitution by applying an additional gentle mixing process such as swirling the reconstitution vessel as described herein in order to provide an optically clear solution.

For the avoidance of doubt, complete reconstitution and complete dissolution provide solutions which are considered to be optically clear. Similarly, near complete reconstitution and near complete dissolution provide solutions which one skilled in the art judges to be very close to complete reconstitution or complete dissolution. The terms reconstitution and dissolution as defined herein are interchangeable. Complete reconstitution/dissolution as defined herein will arise if the dry formulation fully dissolves to produce an optically clear solution. This optically clear solution should exhibit a turbidity of less than 10 NTU when diluted to a protein concentration of 10 mg/ml. Preferably complete reconstitution/dissolution will be obtained either on initial treatment or after gentle swirling as indicated herein.

Foam as defined herein includes, both full and partial layers of foam, or a ring of foam around the surface of the reconstituted solution. The reconstituted solutions prepared according to the methods of the present invention exhibit minimal or no foaming. Minimal foaming includes solutions which are substantially foam free. The presence of a few bubbles at the solution surface or within the solution is not considered to constitute persistent foam and is included within the definition of minimal or no foam or a substantially foam free solution.

Advantageously the method of the present invention provides reconstituted formulations from dry biomolecules, including proteins which exhibit minimal foaming and are free of persistent foam. As indicated hereinbefore, the presence of persistent foam in reconstituted solutions can be related to a reduction in biomolecule or protein integrity i.e. denaturation which is associated with aggregation and the potential for an immune response. Thus the present methods provide a reduced risk of denaturation versus the current methods.

Any suitable dry formulation comprising biomolecules, including dry protein formulation, or combination of dry biomolecule formulations, may be used in the reconstitution method of the present invention including: spray-dried powders or cakes; lyophilised powders or cakes; foams; freeze-spray dried powders; lyophilized protein powders or cakes; precipitated protein powders or cakes; vacuum dried powders or cakes; air-dried powders or cakes; spray dried powders or cakes; and supercritical fluid dried powders or cakes. Suitable dry protein formulations may be prepared according to any of the methods known in the art or as discussed hereinafter.

Thus the reconstitution method of the present invention may be used for the reconstitution of any suitable dry formulation including: spray-dried powders or cakes; lyophilised powders or cakes; foams; freeze-spray dried powders; lyophilized protein powders or cakes; precipitated protein powders or cakes; vacuum dried powders or cakes; air-dried powders or cakes; spray dried powders or cakes; and supercritical fluid dried powders or cakes.

The present invention additionally provides a method as described hereinbefore wherein step (i), or (a) comprises transfer of one or more dry protein formulations into a suitable reconstitution vessel, and wherein said mixture of dry protein formulations may be different dry formulations of the same protein or dry formulations of more than one protein.

Any protein capable of formulation into a dry formulation can be used in accordance with the reconstitution method of the present invention.

Suitable proteins include peptides <5 KDa, small proteins 5-50 KDa, medium proteins, 50-200 KDa and large proteins >200 KDa.

Any one of or combination of the following therapeutic or diagnostic proteins prepared as a dry powder formulation may be used in accordance with the reconstitution method of the present invention: antibodies; non-antibody proteins; immunoglobulins; immunoglobulin-like proteins; growth factors; fusion proteins, chimeric proteins, enzymes; hormones; cytokines; Fc-derivatised proteins or drugs; and recombinant antigens. Suitable antibodies may be polyclonal, monoclonal, native, recombinant, human, humanized, chimeric, multispecific or single chain. Immunoglobulins from classes IgA, IgD, IgE, IgG and IgM may be used. Suitable IgG may be of any isotype including IgG1, IgG2, IgG2Δa, IgG3, and IgG4. Antibody-drug conjugates may also be used. Derivatives of antibodies may also be used and these include the antigen-binding portion produced by recombinant DNA techniques or by enzymatic or chemical cleavage of intact antibodies. Antigen-binding portions include, inter alia, Fab, Fab′, F(ab′).sub.2, Fv, dAb, and complementarity determining region (CDR) fragments, single-chain antibodies (scFv), chimeric antibodies, diabodies and polypeptides that contain at least a portion of an immunoglobulin that is sufficient to confer specific antigen binding to the polypeptide.

Analogs of naturally occurring proteins may be included such as polypeptides with modified glycosylation, polypeptides without glycosylation (unglycosylated), derivatives of naturally occurring or analog polypeptides which have been chemically modified, for example, to attach water soluble polymers (e.g., pegylated), radionuclides, or other diagnostic or targeting or therapeutic moieties) may also be included.

Marketed dry protein formulations which may benefit from the described centrifuge reconstitution process include Synagis® (palivizumab), Herceptin® (trastuzmab), Fuzeon® (enfuvirtide), Xolair® (omalizumab), Raptiva® (efalizumab), and Ilaris® (canakinumab).

Aqueous diluents suitable for use in the reconstitution method of the present invention include: water for injection (WFI), distilled water, deionised water; sterile water for injection (SWFI); and bacteriostatic water for injection (BWFI) i.e. sterile water with a suitable antimicrobial preservative. The aqueous diluent may additionally comprise one or more buffers, surfactants, salts, stabilizers; or mixtures thereof. These may be required to control the tonicity of the reconstituted solution or to stabilise the biomolecule in solution. Buffers, surfactants, salts, and stabilizers suitable for use in the reconstitution method of the invention can be selected from those well-known in the art. The relative amount of aqueous diluent, including where present buffers, surfactants, salts, or stabilizers or mixtures thereon, will be dependent upon the concentration of the target reconstituted protein solution. Selection of suitable buffers, surfactants, salts and stabilizers for use in any particular aqueous diluent will be dependent upon the particular dry protein formulation to be reconstituted.

The applicants have found that when preparing very highly concentrated reconstituted solutions of biomolecule, and particularly protein, that use of an aqueous diluent comprising an aqueous solution of the same biomolecule as that to be reconstituted is advantageous. Thus the present invention additionally provides a method for the reconstitution of dry formulations comprising biomolecules comprising:
i) transfer of a dry formulation comprising biomolecules, preferably a dry protein formulation into a suitable reconstitution vessel, or preparation of a dry formulation comprising biomolecules, preferably a dry protein formulation within a suitable reconstitution vessel;
ii) addition of a suitable quantity of an aqueous solution of the same biomolecule or protein as used in step (i) into the reconstitution vessel
iii) centrifugation of the reconstitution vessel at a suitable relative centrifugal force for sufficient time to obtain complete or near complete reconstitution of the dry formulation into the aqueous diluent and to produce a solution that exhibits minimal foaming;
   wherein the order of steps (i) and (ii) may be reversed or combined providing for the transfer of diluent to the vessel followed by addition of dry formulation, and providing for the transfer of a preformed mixture of dry formulation and diluent to the vessel; and
   wherein the aqueous solution of (ii) may optionally include one or more buffers, surfactants, salts, stabilizers; or mixtures thereof.

The amounts of dry biomolecule, and particularly protein formulation and aqueous diluent used in accordance with the reconstitution methods of the present invention, as described for steps (i), (ii) or (a), will determine the biomolecule, or protein concentration in the resultant solution.

The applicant has also found that the reconstitution methods of the invention become increasingly advantageous as the target concentration of the solution is increased and the volume of the solid dry formulation becomes greater.

High, greater than about 100 mg/ml, and very high, from about 140 mg/ml to about 350 mg/ml, concentrations of protein solutions are especially desirable in the preparation of drug formulations for parenteral administration, such as for example by delivery via a single low volume injection.

In such concentrated and highly concentrated conditions mixing of the solid, dry protein formulation, with the added aqueous diluent becomes harder to achieve using conventional reconstitution protocols.

Using the improved reconstitution methods according to the present invention the difficulties associated with conventional protocols are avoided via centrifugal reconstitution. The applicant has found that effective reconstitution is achieved using the improved methods herein even with very high relative amount of solid in the reconstitution vessel. As illustrated hereinafter the methods of the invention can also be used to reduce the reconstitution time of dry formulations comprising biomolecules by at least 25%. Thus the present invention provides methods for a reduction of at least about 10% to at least about 95% in reconstitution times, more preferably from at least about 25% to at least about 95% reduction in reconstitution times for reconstituted biomolecule solutions prepared by centrifugation when compared to still or continuous or intermittent hand swirling protocols. Most preferably the reconstitution time will be reduced by at least 50% when using centrifugation when compared to still or continuous or intermittent hand swirling protocols. The percentage reduction can be calculated by subtracting the reconstitution time measured using centrifugation from the reconstitution time measured using the still or continuous or intermittent hand swirling protocols, dividing this figure by the reconstitution time using the still or continuous or intermittent hand swirling protocols and multiplying by 100. For example if the reconstitution time using centrifugation is 5 min and that using swirling is 15 min the % reduction is 100×(15-5)/15=66.7%.

Example 1 hereinafter illustrates the advantages of reconstitution of highly concentrated (>200 mg/ml) protein solutions via the present methods in comparison to those provided via conventional protocols.

Thus the methods of the present invention can advantageously be used for the rapid reconstitution of dry protein formulations into protein solutions at high concentrations of greater than about 100 mg/ml, and particularly at very high concentrations in the range of from about 140 mg/ml to about 350 mg/ml, preferably from about 190 mg/ml to about 350 mg/ml and especially from about 200 mg/ml to about 300 mg/ml. In particular the methods of the present invention can provide at least a 25% reduction in reconstitution time for very high protein concentration solutions when compared to still or continuous or intermittent hand swirling protocols. Preferably the methods of the present invention can provide at least a 50% or 90% reduction in reconstitution time for very high protein concentration solutions when compared to still, continuous or intermittent hand swirling protocols. Thus the present invention provides methods for a reduction of from at least about 25% to at least about 90%, more preferably from at least about 50% to at least about 90% reduction in reconstitution times for very highly concentrated protein solutions when compared to still or continuous or intermittent hand swirling protocols.

According to the present invention there are provided rapidly reconstituted foam free solutions of highly concentrated, or very highly concentrated, protein when prepared from corresponding dry protein formulations in accordance with the reconstitution methods hereinbefore.

The relative centrifugal force, applied to the reconstitution vessel in the centrifuge, needs to be sufficient to cause the dry biomolecule formulation to remain immersed or become partially or completely immersed in the aqueous diluent and preferably to accelerate the reconstitution process. The relative centrifugal force (RCF) expressed in units of gravity (times gravity or ×g), increases as the rotor speed of the centrifuge increases. Many microcentrifuges only have settings for speed (revolutions per minute, RPM), not relative centrifugal force. Consequently, a formula for conversion is required to ensure that the appropriate setting is used in an experiment. The relationship between RPM and RCF is as follows:

RCF=(1.118×10⁻⁵) R S²

Where RCF is the relative centrifugal force, R is the radius of the rotor in centimeters, and S is the speed of the centrifuge in revolutions per minute (RCF). Values of RCF are commonly quoted in units of times gravity (×g). As an example, centrifugation of a sample at 5,000 RPM in a microcentrifuge that has a rotor with a radius of 7 cm will deliver a relative centrifugal force of 1,957×g.

The relative centrifugal force (RCF) that is applied should at a minimum be sufficient to ensure that as the reconstitution vessel is rotated in a swing-out rotor substantially all of the diluent-dry formulation mixture remains in the base of the vessel. Application of a sufficient relative centrifugal force (RCF) to a solution is known to lead to the degassing of dissolved air and without wishing to be bound by any particular theory this process may contribute to the observed reduction in reconstitution times. A suitable RCF for reconstituting dry formulations comprising biomolecules is likely to be one that is greater than 10×g. At higher RCF degassing will become more rapid which may explain the observed reduction in reconstitution time of dry formulations with increased RCF. Devices such as blood tube rotators (e.g. Stuart Rotator SB3) are not suitable for use in accordance with the method of the present invention because the RCF they impart is very low (<1×g) and the mixture tumbles from one end (or side) of the vial to the other during each rotation. This leads to detrimental formation of persistent foam as demonstrated in Example 1.

On a small scale suitable relative centrifugal forces can be conveniently achieved by centrifuging the reconstitution vessel in a laboratory centrifuge such as a bench-top centrifuge, or micro-centrifuge. For larger scale processes such as for concentrating protein an industrial centrifuge, such as a decanter centrifuge, may be used. It will be appreciated by those skilled in the art that the centrifuge needs to be balanced during the reconstitution process. This may be achieved by introduction of a vessel of the same mass as the reconstitution vessel containing the formulation and diluent in the opposite position in the centrifuge rotor. Reconstitution of dry formulations comprising biomolecules to form biomolecule or biopolymer solutions, including concentrated protein solutions, in accordance with the method of the present invention can be delivered via centrifugation at a relative centrifugal force of from about 10×g to 10,000×g, and preferably at from about 250×g to about 5000×g. Example 6 illustrates the reduction in reconstitution time for a dry formulation comprising biomolecules when the RCF is increased from 15×g to 1000×g. Thus, the advantages of reduced reconstitution times over conventional swirling methods may become greater as the RCF applied by the centrifuge is increased. However at very high RCF there may be problems caused by excessive compaction of the dry formulation prior to reconstitution or else production of undesirable concentration gradients in the reconstituted solution. As such it is preferred herein to limit the RCF used to less than 5000×g.

For large scale reconstitution of dry formulations comprising biomolecules or biopolymers, prevention of foaming can be more important than reductions in reconstitution time. Thus, reconstitution of dry formulations comprising biomolecules or biopolymers can be advantageously carried out with production of minimal or no foaming by a method comprising centrifugation of the reconstitution mixture at an RCF of 1000×g or 100×g or less.

If the reconstitution vessel is an unusual shape such as a syringe, a dual chamber syringe or a pre-filled syringe a suitable insert may be required to hold it securely within the rotor. The need for and preparation of such a suitable insert is considered to be within the remit of the skilled formulator. Alternatively a bespoke device could be used to apply a suitable centripetal acceleration to the reconstitution vessel. This device should hold the reconstitution vessel firmly in place but allow for easy introduction and removal from the rotor. In the case of a syringe it may additionally be useful for the device to prevent the plunger from moving in the centrifuge. It may also be advantageous if the device allows the syringe to be inverted within the rotor so that full reconstitution can be achieved. This is because it is difficult to swirl the mixture within the chamber of the syringe to remove any material remaining above the level of the solution or else to eliminate a possible concentration gradient.

As the centrifugal forces required for application of the present methods are moderate many types of reconstitution vessels are suitable for use including vials, plastics, multi-well plates, micro-titre plates, eppendorf tubes, trays, bottles, centrifuge tubes, tubes, buckets, bags, sachets, flasks, and syringes. Preferred reconstitution vessels are those compatible with fill-finish protocols for dry protein products such as vials, dual chamber syringes or pre-filled syringes.

Use of prefilled single and dual chamber syringes or cartridges may offer particular benefits to patients as they avoid the need to carry out multiple transfers of diluent or reconstituted solutions between vials and the syringe, thus saving time, avoiding losses and lowering risks. Prefilled single and dual chamber syringes and cartridges may also reduce the amount of expensive therapeutic biomolecule needed per dose because more of the solution can be utilised in the injection. The use of centrifugation as a reconstitution method can be advantageously applied to dual chamber syringes which, prior to reconstitution contain diluent in one chamber and a dry formulation in another chamber. For example, the Lyo-Ject® (Vetter) dual-chamber syringe. Generally these syringes are used with very fast dissolving dry formulations so that, immediately the diluent is allowed to enter the chamber containing the dry formulation, it fully reconstitutes. For dry formulations comprising biomolecules that are required to be reconstituted to high biomolecule concentration the reconstitution would be expected to be very slow because of the extreme difficulty of swirling the diluent-dry formulation mixture within the narrow chamber diameter of a syringe. Furthermore if extensive swirling or shaking is applied it may result in formation of persistent foam and lower the amount of available solution for injection, and lead to a risk of injected air bubbles into the patient. In contrast a dual chamber syringe in which the diluent-dry formulation mixture has been formed can be easily placed in the rotor of a centrifuge by employing a suitable adaptor that holds it and the plunger securely in place. On application of centrifugation for a suitable period of time the mixture may then be reconstituted within the syringe chamber and with minimal formation of foam. This allows many of the advantages of using a prefilled syringe to be retained because all of the solution is rapidly available for injection. In addition a dry formulation comprising biomolecules is likely to be much more stable for long-term storage than a high concentration liquid formulation.

Those skilled in the art will appreciate the same advantages that apply to centrifuging a syringe or dual chamber syringe may also be obtained where the reconstitution vessel for the dry formulation is in the form of a cartridge system or similar device. Example 7 demonstrates the fast reconstitution time and minimal foam arising when a syringe containing a mixture of diluent and a dry formulation comprising biomolecules is centrifuged.

A particular advantage of using centrifugation to enhance the reconstitution of dry formulations comprising biomolecules is that multiple similar or different samples can be easily processed at a time. Thus, the adaptors that fit into the rotors of many centrifuges are very often designed to accommodate many vials at once. Similarly centrifuges are available than can accommodate multi-wall plates. It is therefore possible to simultaneously centrifuge multiple reconstitution vessels containing dry formulation and diluent. This provides for even greater advantages for the centrifugation method over conventional swirling methods of reconstitution. For example a single health-care practitioner could prepare multiple vials of the same or different formulations at the same time. Both the addition of the diluent to the samples and loading and unloading of the samples from the centrifuge may be automated. This may be used to provide a multiplexed reconstitution process which could be designed to be high throughput. The advantage over robot arm based systems is that following reconstitution of a dry formulation comprising biomolecules no or minimal foam is present.

Thus the present invention additionally provides a method for reconstitution of one or more dry formulations comprising biomolecules comprising:
i) transfer of at least one dry formulation comprising biomolecules into a suitable reconstitution vessel, or preparation of at least one dry formulation comprising biomolecules within a suitable reconstitution vessel;

ii) addition of a suitable quantity of an aqueous diluent to the reconstitution vessel(s); and
iii) centrifugation of the reconstitution vessel(s) at a suitable relative centrifugal force for sufficient time to obtain complete or near reconstitution of the dry formulation(s) comprising biomolecules into the aqueous diluent(s) to produce solution(s) that exhibits minimal or no foaming;
   wherein the order of steps (i) and (ii) may be reversed or combined providing for the transfer of diluent to the vessel(s) followed by addition of the dry formulation(s), and providing for the transfer of a preformed mixture of the dry formulation(s) and diluent(s) to the vessel(s) and wherein when more than one dry formulation is used, the biomolecules may be the same or different, and the diluents may be the same or different.

The use of centrifugation is found to reproducibly lower the reconstitution time for suitable dry formulations comprising biomolecules. One problem that may arise is when the volume of liquid diluent used is very low compared to the volume of the dry formulation to be reconstituted. In such cases it is possible for small amounts of the dry formulation to become adhered to the wall of the reconstitution vessel so that it not dissolved into the bulk solution during the centrifugation process and an additional final swirling step is required. This problem can be ameliorated by ensuring that the reconstitution vessel is rotated (or manipulated) as the diluent is slowly added so that the whole vessel wall becomes wetted with dilute diluent before the centrifuge reconstitution process is started. Addition of diluent may for example take 5 to 30 seconds. An alternative approach is to apply a short intense axial rotation to the reconstitution vessel prior to centrifugation as shown in Example 4. One skilled in the art will appreciate that if the reconstitution vessel is cylindrical (such as a vial) then spinning it rapidly around its longitudinal axis will serve to force diluent up the sides of the vessel. This again leads to wetting of the wall with the diluent before is centrifuged. Such a dual step process for reconstitution of a dry formulation with diluent may be combined by using a planetary centrifugal mixer which can simultaneously rotate and centrifuge the reconstitution vessel. Care should be taken however, because rapid axial rotation may produce high sheer forces at the walls of the reconstitution vessel leading to damage of the biomolecule.

For preparation of a sterile product it is preferable for the sterile aqueous diluent to be added aseptically to a sterile dry protein formulation that is sealed within the sterile reconstitution vessel.

The ability to use a simple automated process for reconstituting therapeutic proteins for high concentration delivery without foaming/substantially foam free/with minimal foaming provides a major potential benefit for health care practitioners or self-administering patients with chronic conditions and is a further aspect of this invention. Rather than having to spend time intermittently swirling a vial or syringe until reconstitution is complete, a process that is very hard to carry out reproducibly and which often produces some potentially detrimental foaming, the patient or practitioner is able to simply add the aqueous diluent to the dry protein formulation and place the vial or syringe into a centrifuge for a pre-determined time and at a particular temperature, according to the provided product reconstitution guidelines. A warning signal can be used to alert, for example, the nurse, patient or doctor that reconstitution is complete and on removal from the centrifuge the high concentration protein solution is ready to be administered immediately by, for example, subcutaneous injection. Preferably the centrifuge is provided with the supply of drug and is pre-programmed to carry out the reconstitution using the most appropriate conditions. Alternately a multi-product centrifuge can be used with specific centrifuge reconstitution protocols programmed in for each product. It is also anticipated that the diluent may also be added to the dry protein formulation using an automated device and that the final gentle mixing step may also be automated. This would allow a complete reconstitution process to be carried out without manual intervention.

The reconstitution method of the invention can advantageously be used for the reconstitution of highly temperature sensitive molecules. Thus, using a temperature controllable centrifuge the reconstitution may advantageously be carried out at a low temperature such as 4° C., this would be very difficult to achieve using conventional hand swirling protocols.

In order to be suitable for use in preparing high concentration solutions, and in particular for delivery via parenteral administration, it is desirable that dry protein formulations can be reconstituted in a rapid time but also:
i) show desirable retention of protein integrity and bioactivity following processing and high resistance to degradation on storage;
ii) produce highly concentrated protein solutions on reconstitution exhibiting good clarity and minimal changes to the protein aggregation state to that present prior to protein drying;
iii) produce highly concentrated protein solution on reconstitution in which the concentration of excipients which provides a solution having an osmolality of less than about 800 mOsmol/kg and preferably less than about 600 Osmol/kg at the protein concentration to be administered;

iv) produce highly concentrated protein solutions which exhibit good syringability such that they can be injected using an appropriate bore hyperdermic needle, such as 27G or preferably narrower using a reasonable speed and force. This may be determined by measuring the actual glide force through the target syringe and needle.

Some or all of these requirements may be satisfied by dry protein formulations based on lyophilized powders.

As detailed hereinbefore the reconstituted solutions provided in accordance with the method of the present invention may be advantageously employed to provide biomolecular solutions, suitable for parenteral administration, in numerous situations including: clinical trials; by physicians or care-givers at point of use either in hospitals, clinics or in the home; by self-administration by the patient.

### EXAMPLES

The following Examples are illustrative of specific embodiments of the present invention and are not intended to be limiting thereupon.

### Example 1

Into four identical 6 ml clear glass vials (chrimacol, crimp top, 6-CV) was placed approximately the same measured mass (302 mg) of dry lyophilized albumin powder (Albumin from bovine serum, Sigma Aldrich A7906) and to each was added approximately the same volume (750 μl) of tap water for injection (WFI). Vial A was left undisturbed. Vial B was placed into an ALC Refrigerated Centrifuge PK13OR centrifuge (T535 4-fold swing-out rotor with P510 cups and 4 piece Falcon tube adaptor) and centrifuged for 10 minutes at 2500 rpm at a temperature of about 22° C. To cushion the base of the vial during centrifuging a plug of crumpled paper was placed in the base of the adaptor. The relative centrifugal force applied to the vial was estimated to be about 1000×g. Vial C was left for 30 seconds then gently swirled by hand 10 times and then alternately left for 5 minutes and then swirled 10 more times until reconstitution was complete. Vial D was rotated continuously on a blood rotator (Stuart Rotator SB3) at 20 rpm. The Reconstitution Time was taken as the time period measured from the addition of diluent to the point at which no discernible undissolved or partially dissolved protein powder could be observed by eye in the vial. The additional time for foam and/or trace smears on the vial wall to disappear was not included in the Reconstitution Time.

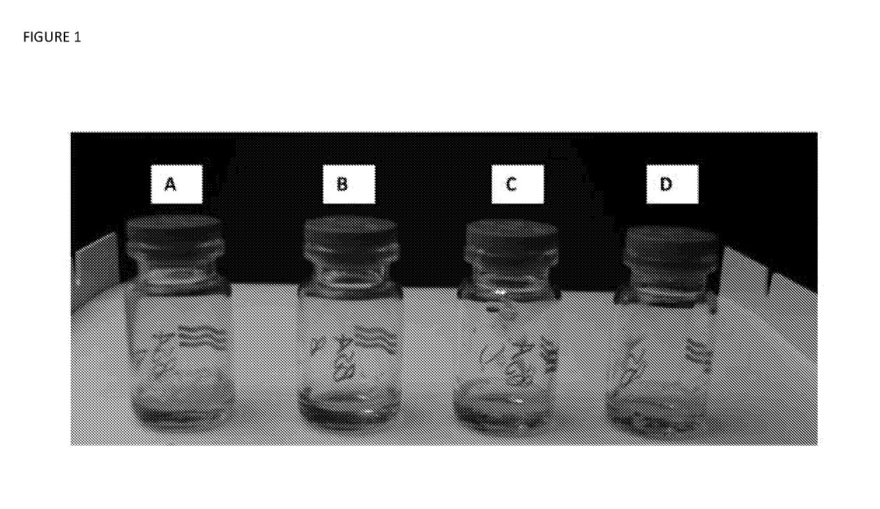

The results of the experiment are described in Table 1 and FIG. 1.

FIG. 1 is a photograph illustrating the vials A, B, C and D from Example 1 and taken 10 minutes after the addition of diluent. The photograph illustrates that reconstitution of the ˜300 mg lyophilized albumin is only complete in vial B which was treated in accordance with the centrifugal method of the present invention. From a comparison of the contents of vials A, B, C and D in FIG. 1 only vial B is essentially foam-free.

TABLE 1

| | Weight of | Volume | Reconsti- | Foam present on |
|---|---|---|---|---|
| | albumin | of WFI | tution | completion of |
| Vial | powder (mg) | (mL) | Time (min) | reconstitution? |
| A | 303.1 | 0.75 | 31 | yes |
| B | 302.1 | 0.75 | 10 | no |
| C | 301.4 | 0.75 | 23 | yes |
| D | 302.0 | 0.75 | 15 | yes |

This experiment demonstrated that when Vial B containing a lyophilized dry protein formulation and aqueous diluent is treated in accordance with the present method, the reconstitution time taken to obtain a clear high concentration protein solutions is surprisingly and advantageously shorter than the reconstitution time required for Vials A, C or D which were treated in accordance with pre-existing protocols. In addition this experiment demonstrates that reconstitution of dry protein powder in accordance with the present method provides a foam-free reconstituted solution.

### Example 2

Example 2 provides a straightforward method to determine whether any example dry formulation comprising biomolecules such as a dry protein formulation is suitable for reconstitution according to the centrifugation method of the invention.

The first step is to reconstitute the dry formulation of interest using any of the conventional methods commonly employed by those skilled in the art. Conveniently this can be carried out at room temperature using a vial or centrifuge tube, as the reconstitution vessel however other vessels may be used. One suitable method or the art involves addition of a defined amount of diluent to the dry formulation in the reconstitution vessel, swirling for 10 seconds leaving the vessel to stand for 5 minutes then re-swirling for 10 seconds and repeating the swirling and standing processes until full reconstitution is judged to have been reached. Preferably the diluent should be slowly added to the dry formulation whilst rotating the reconstitution vessel so that the wall and any material adhering to it become wetted. The point at which full reconstitution is judged to be reached may vary depending on the type of dry formulation under consideration and the application for which it is to be used but should be identifiable reproducibly by someone skilled in the art. The time elapsed from addition of diluent to the dry formulation, to the point at which full reconstitution is attained should be measured and recorded. This is the reconstitution time. The sample should also be carefully examined to determine the extent of the foam that may be present. This can be conveniently recorded by taking a photograph. Of particular relevance is persistent foam which is clearly present as a visible layer on the solution and which remains for longer than about 15 minutes after the sample is judged to be fully reconstituted. If only a few bubbles are observed which do not form a visible layer or ring this is not considered to be persistent foam. If persistent foam is present this should be noted. If desired, the performance of the conventional reconstitution method may be determined more precisely by repeating the reconstitution process three times. The measured reconstitution time can then be averaged and the reproducibility of formation of persistent foam determined. For samples that take longer than a target maximum time to reconstitute using the conventional process (e.g. 90 min) the reconstitution time at which the process was abandoned may be recorded and used for comparison purposes

Typically the scale of the advantage provided by the centrifugation method of the present invention will be sufficiently evident that only one such measurement will be required

The second step of evaluating if a dry formulation comprising biomolecules is suitable for reconstitution by centrifugation is to carry out a centrifugation assessment by placing the reconstitution vessel into a centrifuge and applying a suitable relative centrifugal force to the mixture following the addition of diluent. Centrifuges are common pieces of laboratory apparatus and those skilled in the art will appreciate that it will be necessary to select a rotor and adaptor into which the reconstitution vessel can be placed and to ensure the rotor is balanced. Soft padding can be placed at the bottom of the adaptor to prevent the reconstitution vessel from being damaged. The initial step of the reconstitution process should be carried out identically to that described above for the conventionally reconstituted sample, with the same quantity of diluent added to an identical sample of the dry formulation in the reconstitution vessel. The reconstitution vessel containing the mixture of diluent and dry formulation should then be placed into a centrifuge and centrifuged at a convenient relative centrifugal force (RCF) such as between 50×g and 2000×g, at room temperature. Those skilled in the art will be able to determine from the radius of the centrifuge rotor which speed to apply in rpm using the equation

RCF=(1.118×10⁻⁵) R S²

Where RCF is the relative centrifugal force, R is the radius of the rotor in centimeters, and S is the speed of the centrifuge in revolutions per minute (RPM). Values of RCF are commonly quoted in units of times gravity (×g).

The centrifuge may be stopped and the reconstitution vessel removed from the rotor at 5 or 10 minute intervals to check whether full reconstitution of the dry formulation is judged to have been achieved according to the same criteria used for the conventionally reconstituted samples. If not the reconstitution vessel may be replaced in the rotor and centrifugation continued as required. Once full reconstitution of the dry formulation is obtained the reconstitution time should be recorded and a photograph taken to confirm that minimal or no foam is present. If necessary the experiment can be repeated three times to assess reproducibility and obtain an average reconstitution time.

Dry formulations comprising biomolecules can be considered suitable for reconstitution using the centrifugation method if one or both of the following are observed:
a) the measured reconstitution time for the centrifuged sample(s) is at least 25% lower than the reconstitution time of the sample(s) reconstituted using the conventional method; and/or
b) the sample(s) reconstituted in the centrifuge contains minimal or no foam whilst the sample(s) reconstituted by the conventional method exhibits a layer or ring of persistent foam.

As an example, the reconstitution time of a dry formulation comprising biomolecules was measured to be 20 minutes using the conventional reconstitution method involving stirring and standing. The same dry formulation was found to have a reconstitution time of 14 minutes when centrifuged at a RCF of 1000×g. This dry formulation would therefore be considered suitable for reconstitution by centrifugation since there has been a 30% reduction in the reconstitution time.

As a further example another dry formulation comprising biomolecules had a reconstitution time of 10 minutes when reconstituted by continuous gentle shaking but this resulted in the formation of a persistent layer of foam which was present after a further 20 minutes of standing. When the same dry formulation was reconstituted by centrifugation at a RCF of 1000×g for 10 minutes it was also found to be fully reconstituted but the solution was found to contain just a few bubbles and no persistent foam. This dry formulation would therefore also be considered suitable for reconstitution by centrifugation.

### Example 3

Assessing the Suitability of a Dry Formulation Comprising Biomolecules for Reconstitution by Centrifugation

The suitability of a dry formulation comprising biomolecules for reconstitution using centrifugation was evaluated by comparing the reconstitution time with that obtained using a conventional swirling procedure. The formulation used was a lyophilised preparation of an IgG2 human monoclonal antibody (XmAb). The composition of the XmAb solution used for lyophilisation was 88 mg/ml XmAb, 84 mg/ml trehalose dihydrate, 0.2 mg/ml Polysorbate 80 in 20 mM histidine buffer, pH 5.5. In each case 2.5 ml of solution was loaded into 10 ml capacity vials. The vials were lyophilised using a standard cycle under automatic programming basing on DSC results. Drying was largely complete after 24 hours, but to ensure vial product security overnight the run was allowed to hold for 5 oC before ramping to final secondary drying conditions of 20 C. Vials were back filled with filtered nitrogen to a target of 95% of atmospheric pressure (recorded as 985 mbar), stoppered using the hydraulic ram system the removed from the drier.

To test for reproducibility four samples of the lyophilised IgG2 monoclonal antibody (XmAb) were reconstituted using a conventional swirling method and four samples were reconstituted using centrifugation. For each sample the reconstitution process was started by adding 750 μl of deionised water to the 10 ml vial in which the lyophilised cake containing 200 mg of XmAb was present. For the conventionally reconstituted samples the vial was swirled in an orbital fashion using a radial arc of ˜10 cm for 10 rotations, with the vial base held on the bench, and then left to stand. This process was repeated every 5 minutes until full reconstitution was achieved. For the centrifuged samples the vial was transferred to an adaptor in the rotor of the centrifuge and centrifuged at 750 rpm corresponding to a relative centrifugal force of about 100×g. The results for the swirled samples are shown In Table 3.1 and for the centrifuged samples in Table 3.2.

TABLE 3.1

| | Reconsti- | Reconsti- | Persistent | Final mAb |
|---|---|---|---|---|
| | tution | tution | Foam | concentration |
| | method | Time (min) | present | (mg/mL) |
| Vial 1 | swirl | 120 | yes | 190.77 |
| Vial 2 | swirl | 120 | yes | 183.64 |
| Vial 3 | swirl | 120 | yes | 194.84 |
| Vial 4 | swirl | 180 | yes | 192.73 |
| Average | | 135 | | 190 |
| Standard | | 30 | | 5 |
| Deviation | | | | |
| % RSD* | | 22 | | 3 |

*(STDEV/Average * 100)

TABLE 3.2

| | Reconsti- | Reconsti- | Persistent | Final mAb |
|---|---|---|---|---|
| | tution | tution | Foam | concentration |
| | method | Time (min) | present | (mg/mL) |
| Vial 1 | centrifuge | 32 | no | 194.27 |
| Vial 2 | centrifuge | 40 | no | 196.08 |
| Vial 3 | centrifuge | 40 | no | 205.31 |
| Vial 4 | centrifuge | 43 | no | 200.94 |
| Average | | 39 | | 199 |
| Standard | | 5 | | 5 |
| Deviation | | | | |
| % RSD* | | 12 | | 3 |

*(STDEV/Average * 100)

The results show clearly that this dry formulation is suitable for reconstitution using centrifugation. The average reconstitution time of 39 minutes on applying a relative centrifugal force of 100×g to the vial is about 70% lower than the average reconstitution time of 135 minutes measured when using the conventional swirling reconstitution method. In addition there is no or minimal foam present in the centrifuged samples. The results also demonstrate good reproducibility of reconstitution times when using the centrifugation method with a % RSD=12. This is clearly superior to that for the swirling method where the %RSD=22. A further interesting observation is the reproducibly higher measured protein concentration in the centrifuged samples. This higher retention of soluble protein is surprising and clearly advantageous. Without wishing to be bound to any particular theory it suggests that some protein is lost to the persistent foam observed for high concentration samples reconstituted by conventional swirling methods.

### Example 4

This illustrates the reconstitution of a dry formulation comprising a polysaccharide using centrifugation.

The polysaccharide biopolymer, maltodextrin, obtained by enzymatic conversion of potato starch was supplied by Lyckeby Starkelsen. Two dry sample vials containing 500 mg of powder were diluted with 2.5 mL of deionised water (18.2 MQ). One sample was swirled with the vial bottom maintaining contact with the bench surface, the sample was swirled in an orbital fashion using a radial arc of ˜10 cm using 10 rotations then left to stand. This process was repeated every 5 minutes. The other sample was subjected to centrifugation using a centrifuge operating at 4000 rpm with a rotor radius of 15 cm, which is equivalent to a RCF of ˜2600×g. The swirled sample was not fully reconstituted after >15 minutes whilst the sample subject to centrifugation was fully reconstituted and foam free after 15 minutes.

In order to investigate if the reconstitution process could be further accelerated, a third identical maltodextrin sample was prepared. Prior to insertion into the centrifuge the base of the vial containing the diluent and dry formulation was attached to the top of the axis of the centrifuge rotor and the vial was spun on its longitudinal axis at 4000 rpm for 30 seconds. This step forced the diluent up the side of the vial and wetted the surface. When this pre-treated vial was subjected to centrifugation at ˜2600×g the reconstitution time was reduced to 10 minutes. Axial rotation can therefore be advantageously combined with centrifugation to lower the reconstitution times of dry formulations comprising biomolecules.

Using the inventive method the reconstitution time for maltodextrin powder is reduced by at least 33% in comparison to the time taken using conventional methods.

### Example 5

This illustrates the reconstitution of a spray dried formulation comprising biomolecules using centrifugation.

Spray dried skimmed milk powder was supplied by BD (Becton & Dickinson) and is Difco™ Skim Milk (Ref 232100; Lot 7242704). Two samples were prepared each containing 250 mg diluted in 1 ml of deionised water (18.2MQ). One sample was swirled with the vial bottom maintaining contact with the bench surface, the sample was swirled in an orbital fashion using a radial arc of ˜10 cm using 10 rotations then left to stand for 5 minutes. This swirling and standing process was repeated every 5 minutes. The other sample was reconstituted within a centrifuge operating at 4000 rpm with a rotor radius of 15 cm, which is equivalent to a RCF of ˜2600×g. The swirled sample was not fully reconstituted after >20 minutes whilst the sample subject to centrifugation was fully reconstituted after 10 minutes.

Using the inventive method spray dried milk powder has a reconstitution time that is at least 50% lower than achieved using conventional methods.

### Example 6

### Effect of Different Relative Centrifugal Forces on the Reconstitution Times of a Lyophilised Monoclonal Antibody Formulation

Lyophilised preparations were prepared of an IgG2 human monoclonal antibody (XmAb). The composition of the XmAb solution used for lyophilisation was 88 mg/ml XmAb, 84 mg/ml trehalose dihydrate, 0.2 mg/ml Polysorbate 80 in 20 mM histidine buffer, pH 5.5. In each case 2.5 ml of solution was loaded into 10 ml capacity vials. The vials were lyophilised using a standard cycle under automatic programming, based on differential scanning calorimetry (DSC) results. Drying was largely complete after 24 hours but to ensure vial product security the run was allowed to hold at 5° C. overnight before ramping to the final secondary drying conditions of 20° C. Vials were back filled with filtered nitrogen to a target of 95% of atmospheric pressure and stoppered using a hydraulic ram system before removal from the drier.

Water (Millli-Q, 18.2 MΩ) at room temperature was added as the diluent to the vials containing the dry formulation to start the reconstitution process. In the first low concentration series of four samples sufficient diluent was added to produce a final XmAb concentration of 50 mg/ml. In the second high concentration series of four samples sufficient diluent was added to produce a final XmAb concentration of 200 mg/ml. In each series one sample was reconstituted using a standard conventional reconstitution process (Swirl). Thus, with the vial base maintaining contact with the bench surface, the sample was swirled in an orbital fashion using a radial arc of ˜10 cm using 10 rotations and then left to stand. This process was repeated every 5 minutes till full reconstitution was achieved (>99% of material clearly in solution). The three other samples in each series were placed into a centrifuge immediately following addition of the diluent and centrifuged at a relative centrifugal force of either 15×g, 100×g or 1000×g until full reconstitution was attained. The centrifuge used was an ALC PK13OR centrifuge (T535 4-fold swing-out rotor with P510 cups and 4 piece Falcon tube adaptor). The time from the addition of the diluent to achievement of full reconstitution was measured, the reconstitution time. The results for the two series of four samples are shown in Table 4.

TABLE 4

| | Target | | Reconsti- | Persistent |
|---|---|---|---|---|
| | Concentration | RCF | tution | foam |
| Treatment | (mg/ml) | applied (xg) | time (min) | observed? |
| Swirl | 50 | — | 20 | yes |
| Centrifuge | 50 | 15 | 15 | no |
| Centrifuge | 50 | 100 | 10 | no |
| Centrifuge | 50 | 1000 | 5 | no |
| Swirl | 200 | — | 140 | yes |
| Centrifuge | 200 | 15 | 35 | no |
| Centrifuge | 200 | 100 | 25 | no |
| Centrifuge | 200 | 1000 | 15 | no |

The results demonstrate firstly that the reconstitution time for a dry formulation increases very significantly as the final target concentration is increased. For the samples reconstituted by conventional swirling the reconstitution time increases from 20 min for preparation of the 50 mg/ml solution to 140 min for preparation of the 200 mg/ml solution.

The results show that for both the low and high concentration series there is a clear and surprisingly large beneficial effect of carrying out the reconstitution process in the centrifuge according to the present invention. In each case the reconstitution time is less in the centrifuge and no persistent foam is observed. The reconstitution time is also seen to decrease significantly as the relative centrifugal force applied to the vial is increased. In practical terms this means that as the centrifuge is rotated at higher speed the reconstitution process gets faster. It is possible therefore to choose the RCF which provides the most convenient reconstitution time. It should be noted, however, that higher rotation speeds will place increasing stress on the reconstitution vessel and will also tend to set up a concentration gradient within the reconstituted solution. For this reason the preferred RCF to apply is in the range 250×g and 5000×g.

Using the inventive method a dry formulation of IgG2 human monoclonal antibody (XmAb) can be fully reconstituted in a low concentration solution in about 25% less, or about 50% less, and even up to about 75% less, time than the time taken using conventional methods.

Using the inventive method a dry formulation of IgG2 human monoclonal antibody (XmAb) can be fully reconstituted in a high concentration solution in about 75% less, or about 82% less, and even up to about 89% less, time than the time taken using conventional methods.

### Example 7

The reconstitution of a dry formulation comprising biomolecules is difficult to carry out within a syringe chamber using conventional swirling methods. This is particularly the case where high concentrations of the biomolecule are required because the narrow bore of the syringe chamber makes it difficult to effectively swirl the mixture. Centrifugation can be used advantageously to reduce the reconstitution time but also very importantly to minimise the formation of foam within the syringe chamber. If minimal foam is produced substantially all of the reconstituted solution can be administered. This will minimize the requirement to overfill the syringe leading to waste of expensive therapeutic biomolecules. In this example the same mass of dry precipitated bovine serum albumin powder (330 mg containing 200 mg BSA) was placed either into vials or in the chamber of a syringe (Gerresheimer 2.25mL LL RTF®—Ready to Fill glass syringe) and the same amount of diluent (deionised water, 0.75 ml) was added. In order to hold the syringe firmly in place in the rotor a simple adaptor was made with tissue paper. A comparison was then made of the reconstitution process in either the centrifuged syringe or centrifuged vial or a swirled vial with the results shown in Table 5. It was not feasible to assess the effect of swirling the diluent—dry formulation mixture in the syringe because the chamber volume was taken up almost entirely by the dry powder and the mixture remained stationary when the syringe was rotated.

TABLE 5

| Reconstitution | Reconstitution | Reconstitution | |
|---|---|---|---|
| Vessel | Method | time (min) | Level of foam |
| Syringe chamber | Centrifuge | 10 | none |
| Syringe chamber | Centrifuge | 10 | none |
| Lyo Vial | Swirl | 29 | Lots of foam |
| Lyo Vial | Centrifuge | 10 | none |

This experiment demonstrated that using the centrifugation method the reconstitution time within either a vial or a syringe chamber was the same with no or minimal foam produced. The same sample reconstituted by swirling in a vial gave rise to a lot of foam and the reconstitution time was nearly 3 times greater.

Using the inventive method a dry formulation of precipitated bovine serum albumin can be fully reconstituted in either a syringe chamber or a lyophilised vial in about 10 minutes.

### Example 8

### Preparation of Extremely High Concentration Solutions by Reconstitution Using Centrifugation

Lyophilised XmAb was prepared according Example 6. Lyophilised preparations of bovine serum albumin (BSA were prepared). The composition of the BSA solution used for lyophilisation was 85 mg/ml XmAb, 84 mg/ml trehalose dihydrate, 0.2 mg/ml Polysorbate 80 in 20 mM histidine buffer, pH 5.5. In each case 2.5 ml of solution was loaded into 10 ml capacity vials. The vials were lyophilised using a standard cycle under automatic programming basing on DSC results. Drying was largely complete after 24 hours, but to ensure vial product security overnight the run was allowed to hold for 5° C. before ramping to final secondary drying conditions of 20° C. Vials were back filled with filtered nitrogen to a target of 95% of atmospheric pressure (recorded as 985 mbar), stoppered using the hydraulic ram system the removed from the drier.

Diluent (18.2 MΩwater) was added to the vials of XmAb or BSA containing the dry formulation to start the reconstitution process. Into each vial was added 400 μl, as an estimate of the volume required to achieve 300 mg/mL, as the lyo cake has a significant volume itself. In this experiment one sample was reconstituted using a standard conventional reconstitution process (Swirl) This sample was swirled as follows. With the vial bottom maintaining contact with the bench surface, the sample was swirled in an orbital fashion using a radial arc of ˜10 cm using 10 rotations then left to stand. This process was repeated every 5 minutes till full reconstitution was achieved (>99% of material clearly in solution). The other samples in each series were placed into a centrifuge immediately following addition of the diluent and centrifuged at a relative centrifugal force of either 2700×g until full reconstitution was attained. The centrifuge used was an ALC PK130R centrifuge (T535 4-fold swing-out rotor with P510 cups and 4 piece Falcon tube adaptor. The time from the addition of the diluent to achievement of full reconstitution was measured. The results are shown in Table 6.

TABLE 6

| | | Reconstitution | Measured Protein |
|---|---|---|---|
| Sample | Treatment | Time (min) | Concentration (mg/mL) |
| BSA | swirl | 100 | 289 |
| BSA | centrifuge | 5 | 280 |
| XmAb | swirl | >1380 | 321* |
| XmAb | centrifuge | 45 | 310 |

*to complete the reconstitution process for measurement of concentration the sample was centrifuged

The centrifugation method can clearly be used to accelerate the reconstitution of proteins to very high concentration. For BSA 95% lower reconstitution time was observed. It should be noted that at this high concentration the XmAb sample was extremely viscous and the reconstitution process could not be completed using conventional swirling method. Using centrifugation the reconstitution of XmAb to 310 mg/ml was complete in 45 min.

### Example 9

### Reconstitution of a Dry Biomolecule Formulation with a Biomolecule Solution as Diluent

A high concentration solution of a biomolecule such as a protein can be prepared by using a solution of the biomolecule as the diluent for the same dry biomolecule formulation. This method advantageously avoids the need to produce very high concentration solutions of the biomolecule during the product manufacturing process. The method may be of particular utility where the biomolecule is difficult to concentrate at a large scale because of for example high viscosity or else where the biomolecule exhibits unsatisfactory stability if stored for prolonged period at high concentration. Alternatively the method can be used simply as a convenient route for concentrating a biomolecule solution. Whatever the application, if conventional reconstitution methods, such as swirling and stirring are used to prepare the solution a significant problem is the production of foam.

In this example we show that centrifugation can be used to rapidly reconstitute a lyophilised protein even when a protein solution is used as the diluent and this can be achieved without production of foam. The dry formulation used was lyophilised XmAb prepared according to Example 6 with the dry formulation present in the vials containing ˜200 mg of XmAb.

The diluent used was a 47.6 mg/ml solution of XmAb. 3.8 ml of this XmAb diluent solution was added to dry XmAb formulation in another vial and the mixture was centrifuged at 4000 rpm, RCF=2683×g, using an ALC PK130R centrifuge. A clear, completely foam-free, and fully reconstituted XmAb solution was produced in 7 min. The protein concentration of the XmAb solution produced was 93 mg/ml. The concentration is expected to be slightly lower than 100 mg/ml because of the volume occupied by the excipients and protein. By reducing the amount of XmAb diluent solution added to the dry XmAb formulation the method of the invention enables straightforward production of much higher protein concentrations.

The method of the invention can therefore be advantageously used to rapidly reconstitute a dry biomolecule formulation with a diluent biomolecule solution to form a high concentration biomolecule solution without formation of foam.

1. A method for reconstitution of dry formulations comprising biomolecules comprising the following steps:
(i) transfer of a dry formulation comprising said biomolecules into a suitable reconstitution vessel, or preparation of said dry formulation within a suitable reconstitution vessel;
(ii) addition of a suitable quantity of an aqueous diluent to said reconstitution vessel; and
(iii) centrifugation of the reconstitution vessel at a suitable relative centrifugal force for sufficient time to obtain complete or near complete reconstitution of said dry formulation comprising biomolecules into said aqueous diluent and to produce a biomolecule solution that exhibits minimal or no foaming.

2. The method according to claim 1, wherein the order of steps (i) and (ii) is reversed or combined.

3. The method according to claim 1, wherein the biomolecule solution produced in step (iii) is additionally subjected to axial mixing or gentle mixing before, during or following removal from the centrifuge.

4. The method according to claim 1, wherein the reconstitution vessel is centrifuged in a centrifuge at a relative centrifugal force of from 10×g to 10,000×g.

5. The method according to claim 1, wherein the reconstitution vessel is a vial, syringe, centrifuge tube, multi-well plate, micro-titre plate, eppendorf tube, bottle, tube, bucket, bag, sachet or flask.

6. The method according to claim 1, wherein the reconstitution time is provided in less than 30 minutes.

7. The A method according to claim 1, wherein the biomolecule is a protein,

8. The method according to claim 1, wherein the reconstituted biomolecule solution has a biomolecule concentration of greater than 100 mg/ml.

9. The method according to claim 1 for the preparation of reconstituted solutions suitable for use in clinical trials.

10. The method according to claim 1, wherein the reconstituted biomolecule solution is substantially foam free,

11. The method according to claim 1, wherein multiple samples of the same or different dry formulations comprising biomolecules may be reconstituted simultaneously.

12. The method according to claim 1, wherein the centrifugation of the reconstitution mixture is carried out at a relative centrifugal force of 1000×g or 100×g or less.

13. The method according to claim 6, wherein the reconstitution time is reduced from at least 10% to 95% when compared to the reconstitution times for still or continuous or intermittent hand swirling protocols.

14. A method for the sterile reconstitution of a dry protein via-.
(i) transfer of sterile protein into a sterile reconstitution vessel;
(ii) addition of a suitable quantity of a sterile aqueous diluent to said reconstitution vessel; and
(iii) centrifugation of the reconstitution vessel at a suitable relative centrifugal force and for sufficient time to obtain complete or near complete reconstitution of said sterile dry protein formulation into said aqueous diluent to produce a sterile protein solution that exhibits minimal foaming.

15. A method for the reconstitution of dry protein formulations comprising:
(i) transfer of a dry protein formulation into a suitable reconstitution vessel, or preparation of a dry protein formulation within a suitable reconstitution vessel;
(ii) addition of a suitable quantity of an aqueous solution of the same protein as used in step (i) into said reconstitution vessel; and
(iii) centrifugation of the reconstitution vessel as a suitable relative centrifugal force and for sufficient time to obtain complete or near complete reconstitution of said dry protein formulation into said aqueous diluent to produce a protein solution that exhibits minimal foaming;
wherein the order of steps (i) and (ii) may be reversed or combined providing for the transfer of diluent to the vessel followed by addition of dry protein, and providing for the transfer of a preformed mixture of dry protein and diluent to the vessel;
and wherein said aqueous protein solution of (ii) may optionally include one or more buffers, surfactants, salts, stabilizers; or mixtures thereof.

16. The method according to claim 1, wherein steps (i) and (ii) are combined.
